(19) United States
(12) Patent Application Publication
(10) Pub. No.: US 2007/0038283 A1
(43) Pub. Date: Feb. 15, 2007

(54) Ostial stent and balloon

(76) Inventor: Jihad A. Mustapha, Ada, MI (US)

Correspondence Address: VAN DYKE, GARDNER, LINN AND BURKHART, LLP, 2851 CHARLEVOIX DRIVE, S.E., P.O. BOX 888695, GRAND RAPIDS, MI, 49588-8695, US

(21) Appl. No.: 11/581,676

(22) Filed: Oct. 16, 2006

Related U.S. Application Data
(63) Continuation-in-part of application No. 10/773,709, filed on Feb. 6, 2004.

(51) Int. Cl. A61F 2/06

(52) U.S. Cl. 623/1.11

(57) ABSTRACT

A cardiovascular stent and a stent balloon have portions that provide different degrees of expandability. A distal stent portion has a first degree of expandability to support a vessel, while a proximal stent portion has a second, higher degree of expandability so that it can be radially expanded to form a flange-like structure at an inlet to the vessel. The balloon is configured to deploy the stent and includes distal and proximal balloon portions having different diameters corresponding to the distal and proximal stent portions. The balloon portions may be individually inflated. Optionally, a first stent may be deployed through one branch of a bifurcation, and a second stent may be deployed through a wall of the first stent and into another branch of the bifurcation. The flange on the second stent may position and/or secure the second stent within the first stent.

### CROSS REFERENCE TO RELATED APPLICATION

The present application is a continuation-in-part of U.S. patent application Ser. No. 10/773,709, filed Feb. 6, 2004, which is hereby incorporated herein by reference in its entirety.

### BACKGROUND OF THE INVENTION

The present invention relates to cardiovascular stents and balloons and to methods of using such stents and balloons.

Cardiovascular stents are well known and are widely used in cardiovascular procedures. For example, such a known stent can be inserted into an artery after angioplasty to support the artery at its post-angioplasty size. Wherever used, the stents are delivered to the desired location while mounted on or carried on a deflated balloon to facilitate movement through arteries. When the stent is at the desired location, the balloon is inflated to expand the stent and thereby deploy the stent to support the artery at the desired location.

A typical conventional or known stent 10 is illustrated in FIG. 1. The stent 10 comprises a metallic mesh material and includes a plurality of generally V-shaped struts 12 interconnected in a generally tubular configuration. The V-shaped struts are generally closed when the stent is collapsed (e.g. on a balloon), and the V-shaped struts are generally opened or expanded when the stent is deployed. The spacing, thickness, and strength of the struts can be varied for different applications.

A first exemplary deployment of the stent 10 is illustrated in FIG. 2, in which a first or distal portion 14 of the stent is located within an ostial vessel 40 and a second or proximal portion 16 extends into the primary vessel or aorta 50. A second exemplary deployment of the stent 10 is illustrated in FIG. 3, in which the stent 10 is deployed in an ostial branch 40 extending from a primary vessel 50. In this deployment, the end 18 of the stent 10 is aligned with the wall of the primary vessel 50. This placement is particularly important if the lesion in the ostial vessel is close to the primary vessel 50. If the stent 10 is deployed too far into the ostial vessel, the lesion may not be properly supported, possibly leading to complications.

On the other hand, if the stent 10 extends into the primary vessel 50 (such as shown in FIG. 2), the stent can interfere with the primary vessel or may have further intervention in that region. Consequently, the accurate placement and deployment of the stent is critical. However, even when properly deployed, a single stent may not be capable of supporting all portions of a bifurcation or Y-shaped vessel.

Therefore, there is a need in the art for an ostial stent that overcomes the shortcomings of the prior art.

### SUMMARY OF THE INVENTION

The aforementioned problems are overcome by the present invention, which provides a cardiovascular stent and balloon and method of use that utilizes two different radial expansion or distortion capabilities along the length of the stent and balloon.

According to an aspect of the present invention, a cardiovascular stent and balloon combination comprises a stent and a balloon. The stent comprises a first stent portion having at least a first degree of expandability and a second stent portion having at least a second degree of expandability that is greater than the first degree of expandability. The second stent portion terminates at one end of the stent. The balloon comprises first and second balloon portions, with the second balloon portion being inflatable to a larger diameter than a diameter of the first balloon portion. The balloon is positionable at least partially within the stent, whereby the first balloon portion is positioned at least partially within the first stent portion and the second balloon portion is positioned at least partially within the second stent portion.

The second stent portion is radially expandable so as to form a flange portion at an end of the first stent portion. The first balloon portion may be expandable to a substantially ovoid shape and the second balloon portion may be expandable to a substantially bulbous shape. The first and second balloon portions may comprise individual balloon portions that are individually inflatable via separate inflation tubes.

When expanded, the flange portion of the stent is oriented generally transverse to a longitudinal direction of the first stent portion. The stent comprises struts, with a length of the struts of the first stent portion differing from a length of the struts of the second stent portion. For example, the struts of the second stent portion may be longer than the struts of the first stent portion. The stent may comprise a third stent portion located between the first and second stent portions, with the struts of the third stent portion being generally longitudinally oriented along the stent. The third stent portion provide a transitional portion between the first and second stent portions.

According to another aspect of the present invention, a method of treating a first vessel extending from a second vessel includes providing a stent having first and second stent portions and providing a balloon having first and second balloon portions. The second stent portion is more expandable than the first stent portion, and the second balloon portion is expandable to a larger diameter than a diameter of the first balloon portion. The balloon is positioned at least partially within the stent so that the first balloon portion is at least partially within the first stent portion and the second balloon portion is at least partially within the second stent portion. The stent and balloon are positioned so that the first stent portion is located at the first vessel and the second stent portion is located at the second vessel. The second balloon portion is inflated to expand the second stent portion to form a stent flange. The stent flange is engaged with a wall of the second vessel with the first stent portion and the first balloon portion being within the first vessel. The first balloon portion is inflated to expand the first stent portion to support the first vessel.

According to another aspect of the present invention, a stent balloon includes a first inflatable portion having a first diameter when inflated and a second inflatable portion having a second diameter when inflated. The second diameter is greater than the first diameter, whereby the stent balloon is capable of radially expanding a stent in which it is positioned to two different extents generally corresponding to the first and second diameters. The second inflatable portion is at least partially inflatable independently from the first inflatable portion.

According to yet another aspect of the present invention, a cardiovascular stent includes a first portion having at least a first degree of expandability, a second portion having a second degree of expandability greater than the first degree of expandability, and a third portion between the first and second portions. The second portion terminates at an end of the stent and is expandable to a progressively increased diameter along the second portion and toward the end of the stent. The second portion is expandable to form a flange that is generally transverse to a longitudinal direction of the first portion. The third portion has a third degree of expandability that is different from the first and second degrees of expandability.

The novel stents, balloons, and methods of the present invention provide several advantages. For example, the stents are more securely held in position and therefore are less subject to movement or other complications following deployment. Also, the stents, balloons and methods of the present invention are capable of more fully supporting plaques that are located at and through branches and bifurcations. Further, the stents, balloons and methods of the present invention result in deployment that is more accurate, simple, and effective. Thus, the stents and balloons and methods of the present invention provide enhanced capabilities of deployment of the stents and support of the vessels by the deployed stents.

These and other objects, advantages, purposes and features of the present invention will be more fully understood and appreciated by reference to the description of the preferred embodiments and the drawings.

### BRIEF DESCRIPTION OF THE DRAWINGS

FIG. 1 is a side view of a prior art stent in its deployed or expanded state;

FIG. 2 is a side view of the prior art stent deployed in an ostial vessel extending from the aorta;

FIG. 3 is a side view of the prior art stent deployed in an ostial side branch vessel;

FIG. 4A is a perspective view of the stent of the present invention;

FIG. 4B is a side view of a group of struts of the stent of FIG. 4A;

FIG. 4C is an enlarged view showing the strut structure of a stent of the present invention;

FIG. 5 is a side view of a stent deployed in an ostial vessel in accordance with the present invention;

FIG. 6 is an end view of the deployed stent taken along the line 6-6 in FIG. 5;

FIG. 7 is a side view of the stent deployed in an ostial branch in accordance with the present invention;

FIG. 8 is an end view of the deployed stent taken along the line 8-8 in FIG. 7;

FIG. 9 is a side view of a prior art balloon when inflated;

FIG. 10 is a side view of an ostial balloon of the present invention when inflated;

FIG. 11 is a side view of a stent mounted on a deflated balloon on a guide wire in accordance with the present invention;

FIG. 12A is a side view of the balloon partially inflated to begin deploying the stent in accordance with the present invention;

FIG. 12B is a side view of the balloon fully inflated to complete deployment of the stent;

FIG. 13 is an illustration of a bifurcation with plaques;

FIG. 14 shows conventional angioplasty balloons within the bifurcation;

FIG. 15 shows a stent extending through the bifurcation and into one of the two branches;

FIG. 16 shows an inflated balloon forming an opening in the wall of the stent illustrated in FIG. 15;

FIG. 17 is a sectional view taken along the line 17-17 in FIG. 16;

FIG. 18 is a sectional view similar to FIG. 17 showing the balloon deflated;

FIG. 19 shows the ostial stent of the present invention extending through the opening in the first stent;

FIG. 20 shows the ostial balloon inflated to deploy the ostial stent in accordance with the present invention;

FIG. 21 shows the post-procedure bifurcation supported by both the first stent and the ostial stent;

FIG. 22 is a end view of the ostial stent taken along the line 22-22 in FIG. 21;

FIG. 23 shows a bifurcated stent of the present invention mounted on a pair of guide wires;

FIG. 24 shows the bifurcated stent on the guide wires that extend into the two branches;

FIG. 25 shows the bifurcated stent separated over the two guide wires extending into the branches;

FIG. 26 shows the bifurcated stent in the branches and prior to deployment;

FIG. 27 shows the bifurcated stent fully deployed in the branches;

FIG. 28 is a schematic illustration of the aorta and primary arteries showing the locations where the stents of the present invention might be deployed;

FIG. 29A is an enlarged view showing the strut structure of a stent for use with the ostial balloon in accordance with the present invention;

FIG. 29B is a view showing another strut structure for a stent of the present invention;

FIG. 29C is a view showing another strut structure for a stent of the present invention;

FIG. 29D is a view of the expanded end of the stent of FIG. 29C as it is expanded at the entrance to a side branch or vessel;

FIG. 30 is a side view of another ostial balloon of the present invention when fully inflated;

FIG. 31A is a side view of the balloon partially inflated to begin deployment of the stent;

FIG. 31B is a side view of the balloon fully inflated to complete deployment of the stent;

FIG. 32A is a side view of another ostial balloon and stent of the present invention as it is being inserted into a side branch with the ostial portion of the balloon at least partially inflated and the distal portion being substantially deflated;

FIG. 32B is a side view of the ostial balloon and stent of FIG. 32A with the balloon deployed to expand the stent at and within the side branch; and

FIG. 32C is a perspective view of the deployed stent of FIG. 32B at the entrance to the side branch, showing the expansion of the ostial portion of the stent at and around the side branch entrance.

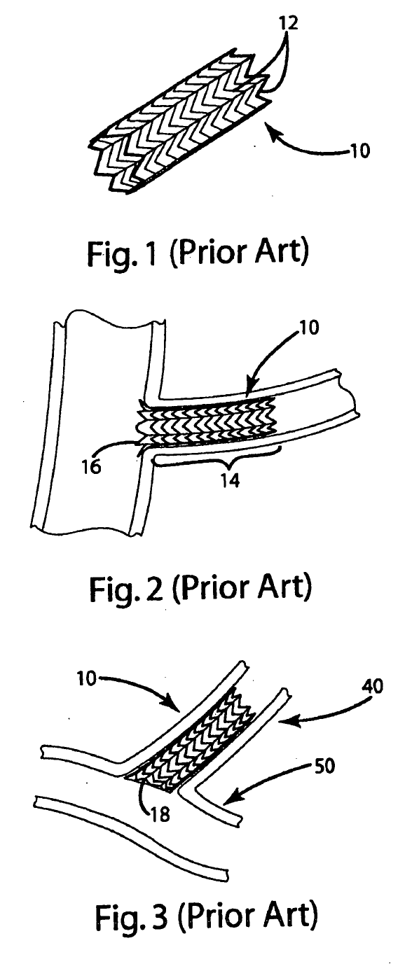

### DESCRIPTION OF THE PREFERRED EMBODIMENTS

I. Ostial Stent

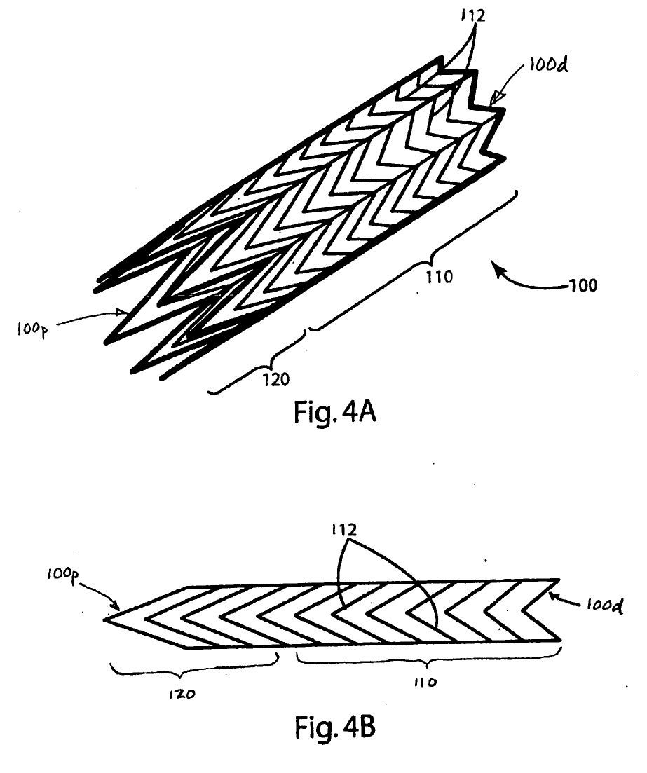

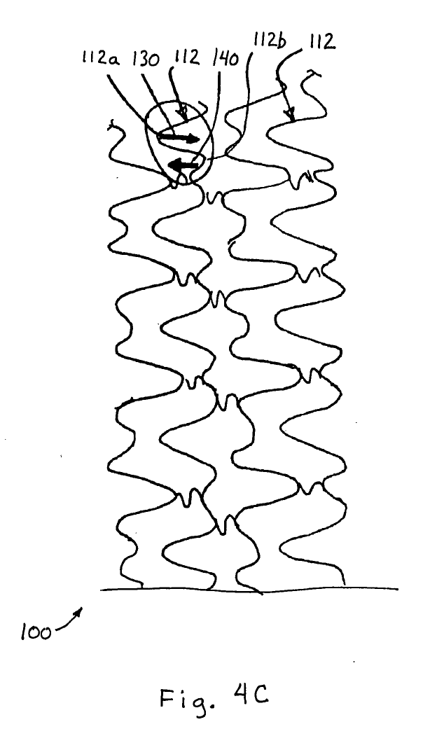

An ostial stent constructed in accordance with a preferred embodiment of the invention is illustrated in FIGS. 4-8 and generally designated 100. The stent 100 comprises a generally distal or support portion 110 and a generally proximal or flange portion 120. The stent 100 comprises a metallic mesh material (or other suitable material) having a plurality of struts that facilitate radial expansion of the stent within the vessel, as discussed below.

The struts 112 of the stent 100 may be uniformly or substantially uniformly spaced along the length of the stent 100. In other words, the distance between any two struts 112 may be substantially equal. However, the lengths of the struts 112 vary along the length of the stent 100. The struts are shortest at or near the extreme distal end 1 00d, and the struts are longest at the extreme proximal end 100p. In a preferred embodiment, the length of each strut is longer than the strut on one side and shorter than the strut on the other side, so that the struts 112 have progressive lengthening and increase in length from the extreme distal end 100d to the extreme proximal end 100p.

Optionally, the distal portion 110 may have struts of one length so that the distal portion may be substantially uniformly expanded to a generally cylindrical shape, while the proximal portion 120 may have struts varying in length and with the strut or series of struts at a junction of the distal portion and the proximal portion being longer than (or having a greater degree of expandability than) the struts of the distal portion. The proximal portion 120 thus may be radially expanded to a larger size than the distal portion to enhance the expansion and implementation of the stent, as discussed below.

In the illustrated embodiment, each of the struts is generally V-shaped. Because the struts vary in length, the Vs form different angles depending on the length of the strut. When the stent 100 is collapsed (as illustrated in FIG. 10), the relatively short struts in the distal portion 110 form relatively large angles, and the relatively long struts in the proximal portion 120 form relatively small angles. Although shown and described as generally V-shaped struts, clearly the stent may include struts of different shapes, such as generally U-shaped struts or other suitable shape or shapes that allow the desired or appropriate degree of expansion and contraction of the stent, while remaining within the spirit and scope of the present invention. Further, although shown and described as having longer struts at the proximal or flange portion of the stent, the struts of the flange portion and distal portion may be otherwise configured and arranged to provide for greater expandability of the flange portion relative to the distal portion, while remaining within the spirit and scope of the present invention.

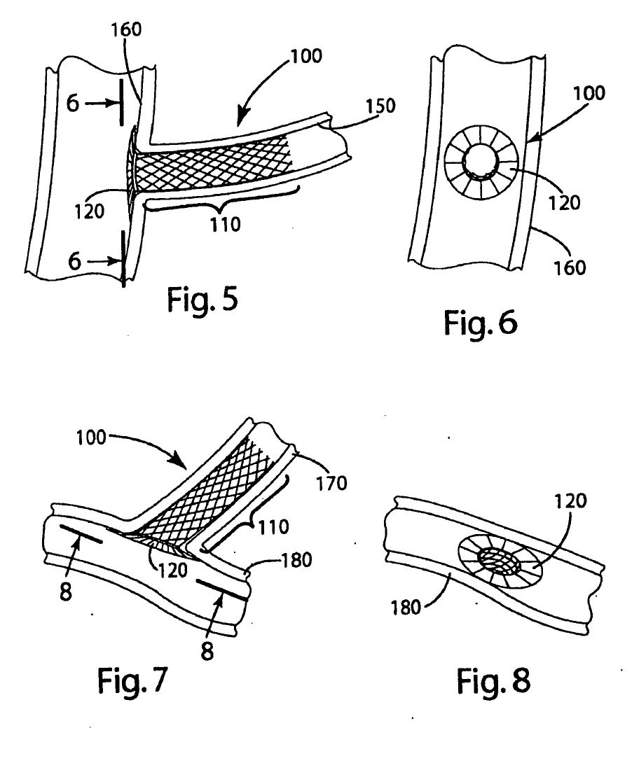

Because the struts are longer (or otherwise more expandable) at the proximal portion 120 as compared to the struts of the distal portion 110, the two portions 110 and 120 therefore are capable of different radial expansions or distortions as the stent is radially expanded and the struts partially straighten out during such expansion. The distal portion 110, with the shorter struts, thus may be expandable to a degree in the range of prior art stents and may be expanded to a generally cylindrical shape. Consequently, the distal portion 110 is suited for supporting a vessel, such as shown in FIGS. 5-8. The proximal portion 120 is expandable to a far greater degree than that of the distal portion 110 due to the expansion or generally straightening of the larger or longer struts (or otherwise configured struts) at the distal portion.

Because the struts of the proximal portion 120 are progressively increasing in length toward the proximal end 100p, the struts may provide a flared-out flange arrangement at the end of the distal portion 110 as the struts are substantially straightened out during expansion of the stent. As shown in FIGS. 5-8, the proximal portion 120 may be radially expanded until it is generally perpendicular to the longitudinal axis of the stent, thereby forming a flange on or at an end region of the distal portion 110 of the stent.

FIG. 4C is an enlarged view showing more specifically the presently envisioned structure for the struts 112. Arrows 130 and 140 show the direction of movement of the strut portions 112a and 112b respectively as the stent expands. The two directions are opposite one another and the struts thus generally straighten or partially straighten during expansion of the stent. The specific structure shown in FIG. 4C enables the struts 112 to be substantially uniformly spaced (i.e. the distance between adjacent struts is substantially constant), while the varying length of the struts allows for different degrees of radial expansion of the stent in response to partial straightening of the different length struts.

The disclosed stent 100 is but one example for constructing a strut having different degrees of expandability or distortion along its length. The length of the struts can vary in a fashion other than as described. Further, other techniques for providing different portions or areas of expansion or distortion will be known to those skilled in the art.

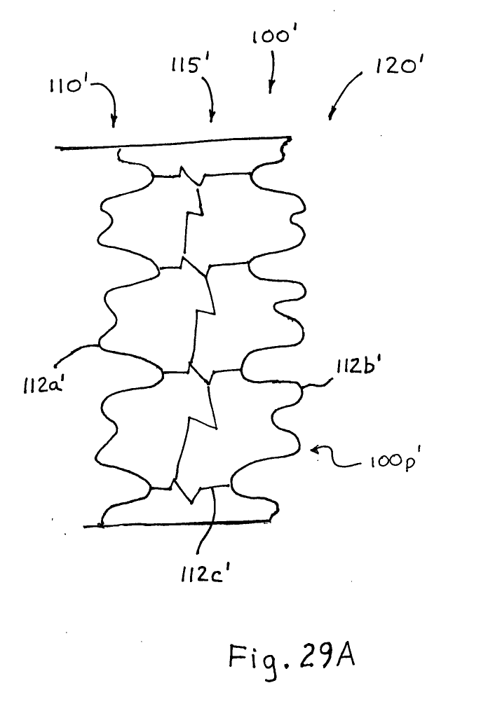

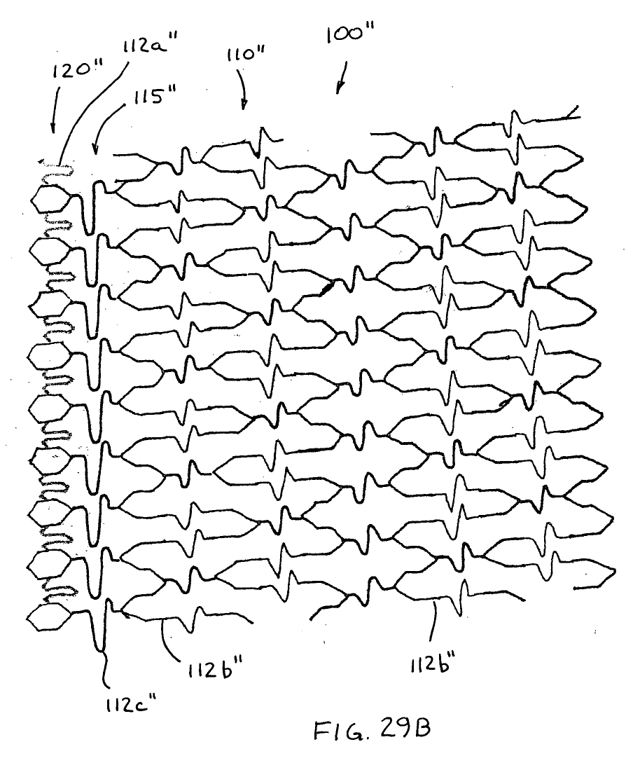

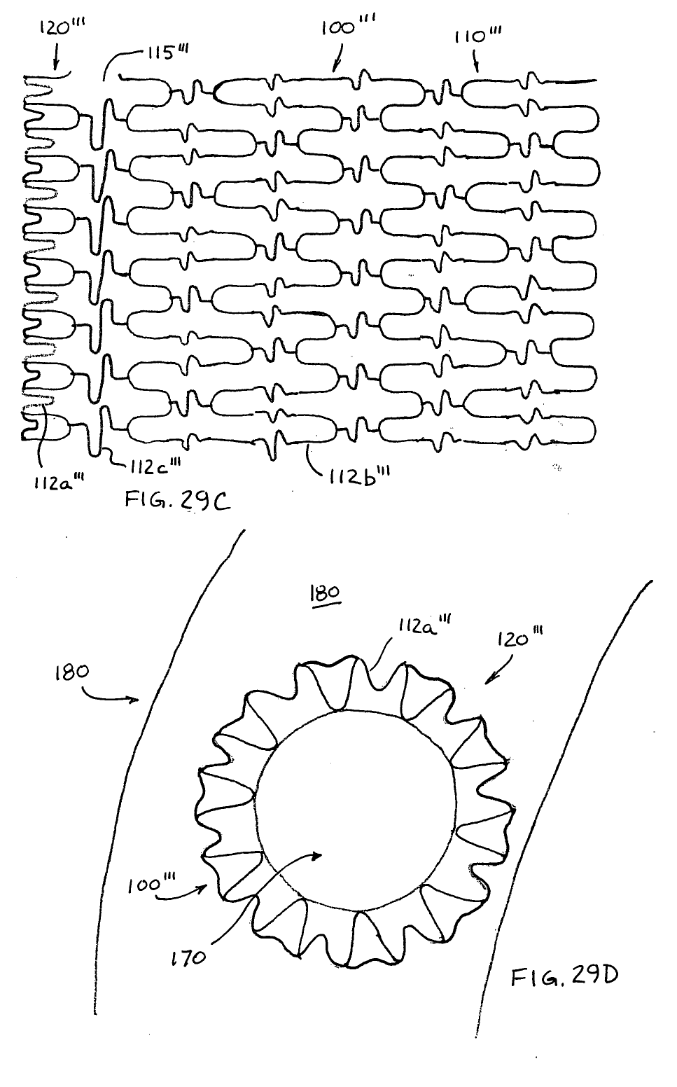

Optionally, the stent may include a transition section between the smaller strut distal portion and the larger strut proximal portion so that expansion of the proximal stent portion will have a reduced or minimal effect on the expansion/deployment of the distal stent portion. For example, and with reference to FIG. 29A, a stent 100′ includes a proximal or flange portion 120′ and a distal or support portion 110′ and a transitional section or portion or region 115′ therebetween. The struts 112a′ of the proximal portion 120′ may be progressively lengthened toward the proximal end 100p′ of the stent 100′ (for example, each strut or set of struts around and along the proximal portion 120′ is longer than an adjacent strut or set of struts toward the transitional portion 115′). The struts 112b′ of distal portion 110′ may be of similar lengths (such as for a generally cylindrical deployment of the support or distal portion of the stent) or may have progressively lengthening or otherwise varying struts therealong, depending on the particular application of the stent.

As can be seen with reference to FIG. 29A, the struts 112c′ of transitional portion 115′ may be arranged generally longitudinally along the stent to allow for enhanced flexibility of the transitional portion 115′. Such an arrangement further allows for substantial or full expansion of the proximal portion 120′ with reduced or minimal effect on the expansion of the distal portion 110′. The proximal portion 120′ thus may be radially expanded to form or establish a flange portion of the stent that is generally orthogonal or transverse to a longitudinal direction of the support portion of the stent without increasing the expansion or affecting the expansion of the support portion at the junction of the support portion and the flange portion. The support portion thus may be substantially cylindrical along its entire length (or close to its entire length) without any flaring or expansion of the end region of the support portion (that may be positioned within the ostial vessel or branch when the stent is implemented and deployed at a targeted vessel) and at or near the flange portion.

Optionally, other strut configurations and arrangements may be implemented while remaining within the spirit and scope of the present invention. For example, and with reference to FIG. 29B, a stent 100″ includes a proximal or flange portion 120″ and a distal or support portion 110″ and a transitional section or portion or region 115″ therebetween. The struts 112a″ of the proximal portion 120″ may be longer or more radially expandable than the struts 112b″ of distal portion 110″ (and may be progressively lengthened toward the proximal end of the stent as described above). The struts 112b″ of distal portion 110″ may be of similar lengths to one another (such as for a generally cylindrical deployment of the support or distal portion of the stent) or may have progressively lengthening or otherwise varying struts therealong, depending on the particular application of the stent. As shown in FIG. 29B, the struts 112c″ of transitional portion 115″ may be arranged generally longitudinally along the stent to allow for enhanced flexibility of the transitional portion 115″, such as described above. The proximal portion 120″ thus may be radially expanded to form or establish a flange portion of the stent that is generally orthogonal or transverse to a longitudinal direction of the support portion of the stent without increasing the expansion or affecting the expansion of the support portion at the junction of the support portion and the flange portion.

Optionally, and with reference to FIGS. 29C and 29D, a stent 100′″ includes a proximal or flange portion 120′″ and a distal or support portion 110′″ and a transitional section or portion or region 115′″ therebetween. The struts 112a′″ of the proximal portion 120′″ may be longer than the struts 112b′″ of distal portion 110′″ (and may be otherwise configured and/or arranged to allow for greater expansion of the proximal portion as compared to the distal portion). The struts 112b′″ of distal portion 110′″ may be of similar lengths (such as for a generally cylindrical deployment of the support or distal portion of the stent) or may have progressively lengthening or otherwise varying struts therealong, depending on the particular application of the stent. As shown in FIG. 29C, the struts 112c′″ of transitional portion 115′″ may be arranged generally longitudinally along the stent to allow for enhanced flexibility of the transitional portion 115′″, such as described above. As can be seen with reference to FIG. 29D, the proximal portion 120′″ thus may be radially expanded to form or establish a flange portion of the stent that is generally orthogonal or transverse to a longitudinal direction of the support portion of the stent without increasing the expansion or affecting the expansion of the support portion at the junction of the support portion and the flange portion. The proximal portion 120′″ thus may be located at the wall 180a of the primary vessel 180 and generally at and around the opening to the side branch or vessel 170 where the distal portion of the stent is located.

Optionally, and desirably, the stent may include a circumferential marker (not illustrated), which may be located on or at the outer wall of the stent (such as in a conventional fashion) to assist the physician in properly locating the stent during the procedure. Such markers are known in the art and need not be discussed in detail herein.

The stent of the present invention thus provides significant flexibility, accommodates angled vessels without losing the integrity of the stent, and enables deployment of the stent in vessels whose diameters vary along the location of the stent. The stent may have two separately or individually expanded portions to enhance the location of and deployment of the stent at an ostial branch or vessel.

For example, FIGS. 5-6 illustrate the deployment of the stent 100 within an ostial branch 150 extending from a primary vessel 160 (although the following discussions refer to stent 100, the discussions and applications apply equally to stents 100′, 100″ and 100′″, and to other suitably constructed stents). The distal portion 110 of the stent is located within the ostial branch 150, and the proximal portion 120 (with the longer struts and thus capable of a greater degree of radial expansion) is located at the wall of the primary vessel 160 and generally at the opening of the ostial branch 150 at the wall of the primary vessel. The proximal portion 120, when expanded, is flared outwardly to form a flange at or against the interior wall of the primary vessel 160. When so deployed, the distal portion 110 supports the vessel 150. The flange 120 assists in maintaining the stent 100 in proper position relative to the primary vessel and ostial branch subsequent to the catheterization procedure.

Optionally, and as shown in FIGS. 7-8, the stent 100 may be deployed in another ostial branch 170 extending from a primary vessel 180. The distal portion 110 of the stent 100 is located within the ostial branch 170 to support the branch. The proximal portion 120 is located at the wall of the primary vessel 180 and generally at the opening of the ostial branch 170 at the wall of the primary vessel. The proximal portion 120, when expanded, is flared outwardly to form a flange against the interior wall of the primary vessel 180. The marker may be aligned with the wall of the primary vessel 180 to properly locate the stent at the branch.

The ostial stent of the present invention can be mounted on a conventional balloon for deployment in “straight” vessels. The struts for straight stents would have an angulation in the approximate range of 45 degrees to 55 degrees in comparison to the approximate range of 30 degrees to 60 degrees in the above described ostial stent. The greater angulation provides appropriate support for proximal vessel walls. Optionally, and preferably, the ostial stent of the present invention is implemented in conjunction with an ostial balloon that has different degrees of expansion therealong, so as to deploy the stent with different degrees of radial expansion between the proximal portion and the distal portion of the stent, as discussed below.

II. Ostial Balloon

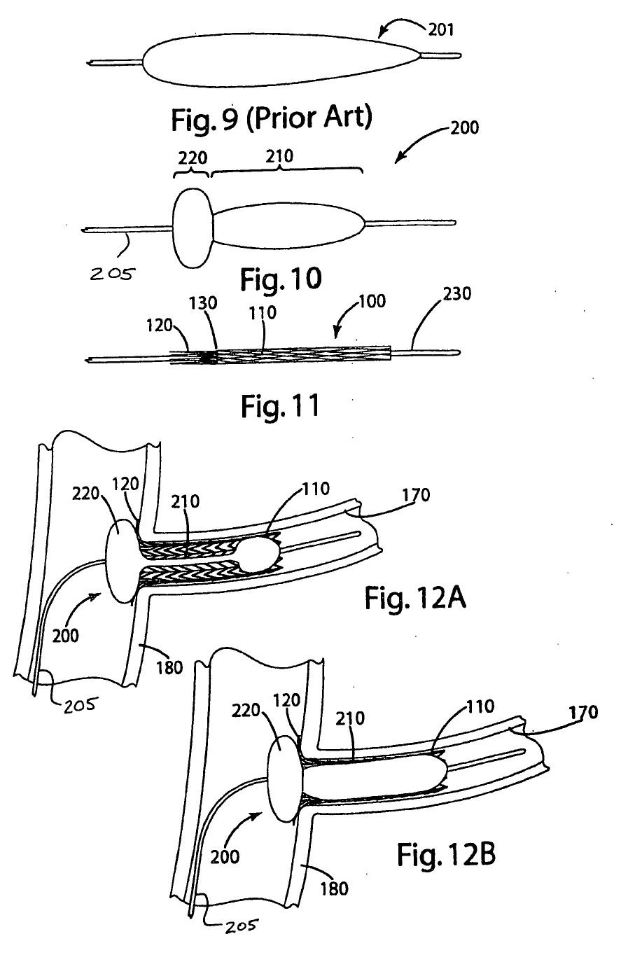

A prior art stent balloon 200 is illustrated in FIG. 9. The prior art balloon is ovoid or cigar-shaped when inflated as illustrated in FIG. 9. As is well known in the art, a stent may be mounted over the deflated balloon before the stent/balloon combination is delivered to the desired location. When the stent is properly positioned, the balloon is inflated (such as via an inflation line or tube) to expand or deploy the stent into its operative configuration. The prior art balloon 200 may be capable of deploying the stent into a generally cylindrical shape.

However, such a prior art balloon 200 cannot expand the proximal portion 120 of the stent 100 into its flange-shaped configuration while inflating the distal portion 110 of the stent into its generally cylindrical shape.

An ostial balloon constructed in accordance with the present invention is illustrated in FIGS. 10-12 and generally designated 200. The balloon 200 includes a first longitudinal portion or distal portion 210 and a second longitudinal portion or proximal portion 220. The distal portion 210 when fully inflated (FIG. 12B) is generally ovoid, while the proximal portion 220 is generally bulbous when fully inflated, and has a diameter that is substantially greater than that of the distal portion 210. In the illustrated embodiment of FIGS. 10-12, the distal portion 210 and the proximal portion 220 are integrally connected to one another, and may be inflated via the single inflation line 205. Consequently, inflation of the balloon 200 results in inflation of both portions 210 and 220. Optionally, the materials or elasticity or expandability of the portions may be selected to be different from one another so that one portion (such as the proximal portion 220) may be inflated or partially or substantially inflated before inflation of the other portion (such as the distal portion 210). Such a varied degree of inflation allows for positioning of and inflation of the proximal portion to deploy the proximal portion of the stent prior to deployment of the distal portion of the stent, which eases the positioning of the stent at the vessel. Methods and techniques for fabricating the balloon will be known to those skilled in the art.

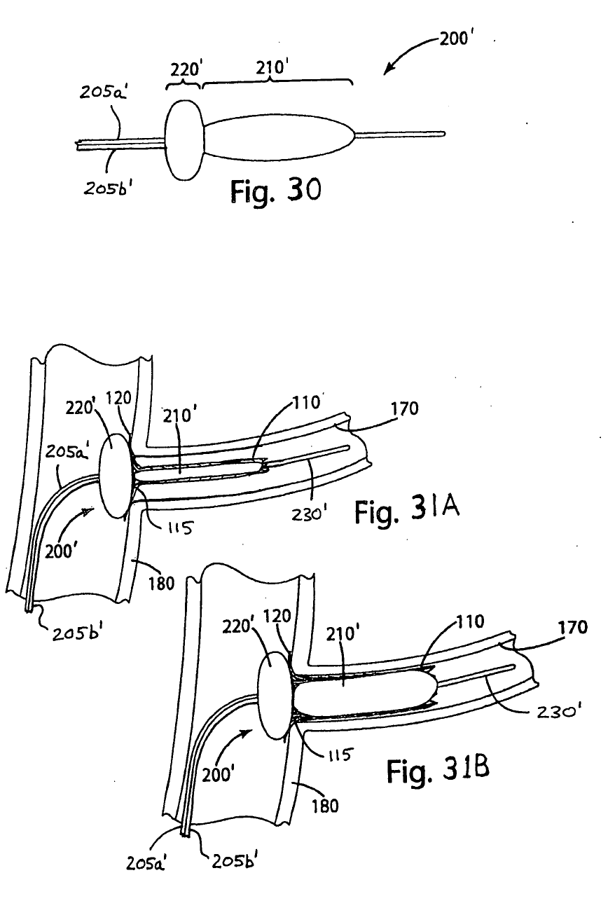

Optionally, the balloon may have two separate chambers or portions that are inflated separately or individually to provide the desired expansion of the stent portions to deploy the stent at the vessel. For example, and with reference to FIGS. 30, 31A and 31B, a dual chamber balloon 200′ may comprise a proximal or flange portion 220′ and a distal or support portion 210′ that are individual portions or chambers. In the illustrated embodiment, the proximal portion 220′ is inflatable via an inflation tube 205a′, while the distal portion 210′ is separately or individually inflatable via a second inflation tube 205b′. The second inflation tube 205b′ may pass through the proximal portion 220′ to the distal portion 210′ or may be positioned or routed around or along (and may be retained at or secured to) an exterior of the proximal portion 220′ to the distal portion 210′. The inflation tubes 205a′, 205b′ may be connected at their inlet ends (not shown) to separate inflation ports (such as a proximal port and a distal port at an inflation control or machine) that are separately or individually controlled to selectively inflate the desired or appropriate portions of the balloon.

The proximal portion 220′ and the distal portion 210′ of balloon 200′ may otherwise be substantially similar in shape or construction as the proximal portion 220 and distal portion 210 of balloon 200, discussed above. For example, the proximal portion 220′ may be inflatable to a generally bulbous shape so as to deploy the proximal or flange portion of the stent at the wall of the main vessel and generally around the opening of the ostial or branch vessel, while the distal portion 210′ may be inflatable to a generally cylindrical or ovoid shape to deploy the distal or support portion of the stent along the ostial or branch vessel.

Because the portions 220′, 210′ are individually inflatable via separate or individual inflation lines, the elasticity or expandability of the portions or chambers may be the same or different, depending on the particular application and without affecting the present invention.

III. Procedure Using the Stent and Balloon of the Present Invention

When implementing and deploying the stent of the present invention, the stent is positioned or mounted over and along a balloon on a guide wire. The guide wire is inserted into the vessel and, when positioned at the appropriate vessel, the balloon and stent arrangement is moved along the guide wire until the stent is properly positioned or located at the vessel that is to be expanded or supported by the stent.

For example, and with reference to FIGS. 11, 12A and 12B, the stent 100 and balloon 200 may be positioned at a vessel for deployment. The stent 100 is shown in FIG. 11 in its collapsed condition mounted over a balloon (not visible) on a guide wire 230. As is well known to those skilled in the art, the guide wire 230 is used to guide the stent 100 to the deployment location. The inflation line for the balloon may be routed along the guide wire between the balloon and the entry point of the patient.

FIGS. 12A and 12B illustrate the ostial balloon 200 at two different stages of inflation to deploy the stent 100. More particularly, FIG. 12A shows the first balloon portion partially inflated, and FIG. 12B shows the first balloon portion fully inflated. As shown in FIG. 12A, the initial inflation of the balloon results in the distal end of the distal portion being inflated while the proximal portion is inflated (which may be due to a selected elasticity or expandability of the stent and/or the balloon at the different regions of the stent and/or balloon so as to control the expansion of the balloon and the stent along the stent). As inflation continues, the balloon inflates toward the center from the opposite ends. When fully inflated (such as shown in FIG. 12B), the distal portion 210 of the balloon deploys the distal portion 110 of the stent 100. The described inflation sequence traps all plaques within the stent, preventing a distal embolization. Similarly, the proximal portion 220 of the balloon deploys the proximal portion 120 of the stent 100. Because the distal portion 210 may be substantially similar in size and shape to the prior art stent 201 (see FIG. 9), this portion of the balloon properly deploys the supportive distal portion 110 of the stent 100, such as within the ostial branch 170. Also, because the proximal portion 220 of balloon 200 has an inflated diameter substantially greater than the inflated diameter of the distal portion 210, and further because of the bulbous shape of the proximal portion when it is inflated, the proximal portion 220 forms or expands the proximal portion 120 of the stent into a flange against the wall of the main branch 180 and generally around the opening of the side or ostial branch.

Thus, the guide wire may be inserted into a vessel to be supported and the deflated balloon and stent carried thereon may be moved along the guide wire to locate the balloon and stent at the ostial branch or vessel. The balloon may be inflated to deploy the stent at the vessel. For example, the proximal portion 220 of balloon 200 may be initially inflated or partially inflated to deploy the proximal portion 120 of stent 100 so as to form or establish the flange of the stent at the wall of the main branch 180. When the flange is so established and the stent is located at and partially in the ostial branch 170 (with the flange engaging the wall of the main branch and substantially around the opening into the ostial branch), the balloon may be further inflated to inflate the distal portion 210 of balloon 200 to deploy the distal portion 110 of stent 100, which causes the radial expansion of the distal portion 210 to engage and support the walls of the ostial branch 170.

With respect to the dual chamber balloon 200′, the procedure for implementing and deploying the stent at the ostial branch of the vessel may be substantially similar to that described above with respect to the balloon 200. In such an application, the proximal portion 220′ of balloon 200′ may be initially inflated or partially inflated via inflation line 205a′ to deploy the proximal portion or flange 120 of stent 100 (such as shown in FIG. 31A). The balloon and stent combination may then be advanced along the guide wire 230′ until the flange 120 contacts the wall of the primary vessel (with the deflated distal portion 210′ of balloon 200′ and the undeployed distal portion 110 of stent 100 extending into and along the ostial branch). The distal portion 210′ of balloon 200′ may then be inflated or partially inflated via inflation line 205b′ to deploy or expand the distal portion or support portion 110 of stent 100 within the ostial branch (such as shown in FIG. 31B), such as in a similar manner as described above. Other deployment or inflation orders or procedures (for example, the distal portion may be inflated or partially inflated prior to inflation of the proximal portion) may be implemented while remaining within the spirit and scope of the present invention. Optionally, the stent may include a transitional region or portion 115 at the junction between the proximal portion and the distal portion to allow for the expansion of the proximal portion with a reduced effect on the distal portion, such as discussed above.

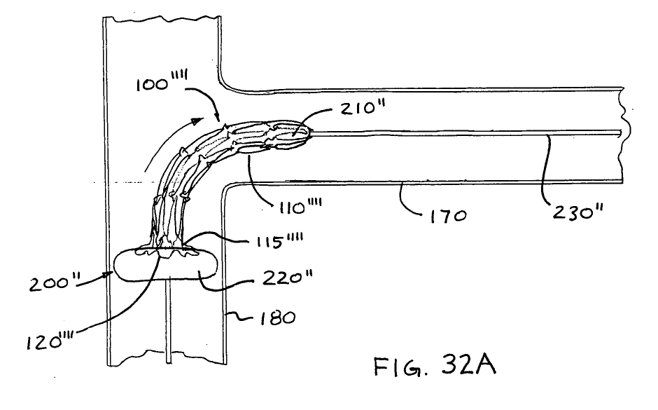

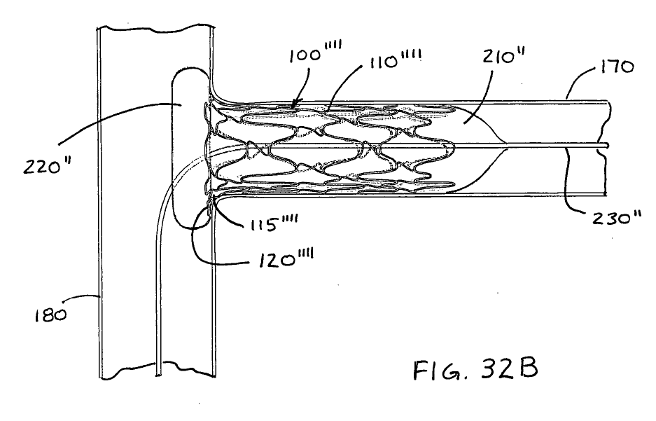

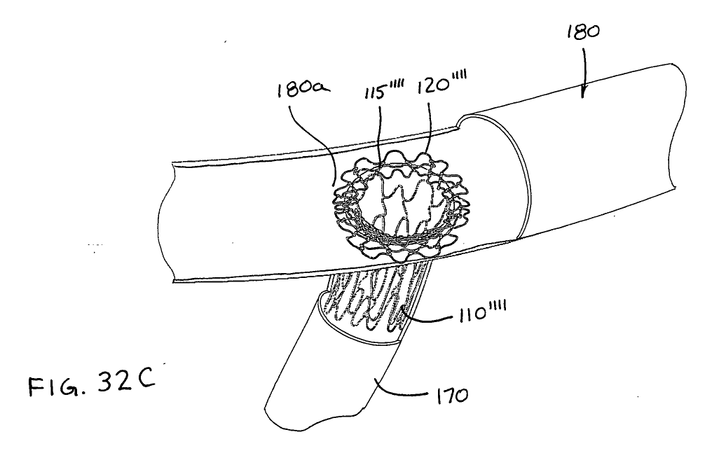

Optionally, and with reference to FIGS. 32A-C, a stent 100″″ and balloon 200″ may be inserted and deployed within a side branch vessel 170 in a similar manner as described above. More particularly, and as shown in FIG. 32A, the proximal portion 220″ of balloon 200″ may be initially inflated or partially inflated deploy the proximal portion or flange 120″″ of stent 100″″ within the primary vessel 180. The balloon and stent combination may then be advanced along the guide wire 230″ with the distal portion 210″ of balloon 200″ being substantially uninflated. As can be seen with reference to FIGS. 32A and 32B, the balloon and stent are advanced along the guide wire until the flange 120″″ contacts the wall of the primary vessel (with the deflated distal portion 210″ of balloon 200″ and the undeployed distal portion 110″″ of stent 100″″ extending into and along the ostial branch). The distal portion 210″ of balloon 200″ may then be inflated or partially inflated to deploy or expand the distal portion or support portion 110″″ of stent 100″″ within the ostial branch (such as shown in FIG. 32B), with the transition section or portion 115″″ of stent 100″″ adapting or configuring the stent to conform along the walls of the vessels between the proximal and distal portions of the stent. After the balloon is inflated and the stent is deployed, the balloon may be deflated and removed from the stent and vessels. When the stent 100″″ is deployed, the flange or proximal portion of the stent is at the wall 180a of the primary vessel 180 and substantially flush along the wall at the opening to the side branch or ostial branch or vessel 170, while the distal portion of the stent is within the side branch to maintain the side branch in its expanded/supported state.

Optionally, a dual chamber balloon or balloon with a bulbous portion and a cylindrical or ovoid portion and dual expandability stent of the present invention may be implemented as an aortoilliac balloon and stent for expanding a vessel during an aortoilliac procedure. In such a procedure (or any similar procedure where the vessels may be accessed at a branch vessel with the balloon and stent advanced along the branch vessel toward and partially into the primary vessel or aorta), the balloon and stent may be reversed, so that the proximal portion of the balloon (the portion closest to the entry point into the patient's body for the guide wires, balloon and stent) is substantially cylindrical or ovoid and within the generally cylindrical-shaped stent portion, and the distal portion of the balloon is substantially bulbous and at or within the increased expandability flange portion of the stent. Thus, the balloon and stent may be advanced along the branch vessel until the distal or bulbous portion of the balloon (and the distal or flange portion of the stent) is past the branch of the vessel and into the primary or other vessel. The distal portion (bulbous portion) of the balloon may then be inflated to expand the distal or flange portion of the stent, and the balloon and stent may be pulled or partially retracted until the flange portion of the stent engages the wall of the other vessel, thereby locating and maintaining the balloon and stent at the appropriate location. The proximal portion (the ovoid-shaped portion) of the balloon may then be inflated to expand the proximal or support portion of the stent, whereby the stent is fully deployed to support the branch vessel in a similar manner as described above.

Therefore, the balloon and stent of the present invention provides enhanced positioning of the stent at a branch vessel, and provides such positioning when advanced or retracted in either direction. The portions or chambers of the balloon may be independently and selectively inflated to first expand the flange portion of the stent to assist in properly locating the stent, and then to expand the support portion of the stent to properly deploy the stent within the targeted vessel.

IV. Procedure Using Both Conventional Stent and the New Ostial Stent

The ostial stent 100 can be used in conjunction with a conventional stent 10 (or another ostial stent) to fully support a bifurcation or branch in which an incoming vessel and two outgoing vessels meet in a Y-shape junction. Further, the procedure results in a combination device that fully support all areas within and through the bifurcation.

Optionally, aspects of other stent and balloon assemblies or arrangements (such as the stents described in U.S. provisional applications, Ser. No. 60/730,336, filed Oct. 26, 2005 (Attorney Docket MUS02 P-100); and Ser. No. 60/799,099, filed May 10, 2006 (Attorney Docket MUS02 P-100A), which are hereby incorporated herein by reference in their entireties) may be implemented with the stent and balloon of the present invention.

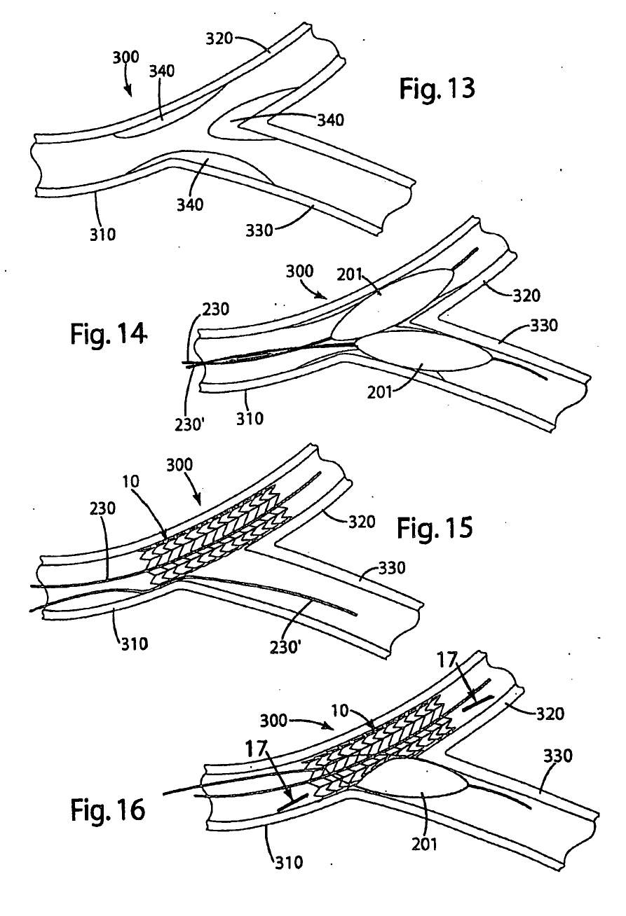

An exemplary bifurcation 300 is illustrated in FIG. 13. The bifurcation includes an incoming or main branch or vessel 310 and two outgoing or extending branches or vessels 320 and 330. As disclosed, plaques 340 may exist in any or all of the variety of areas illustrated.

The first step in treating the plaques is conventional kissing angioplasty as illustrated in FIG. 14. During such a procedure, a pair of balloons, such as conventional balloons 201, may be inserted into the vessel using guide wires 230 and 230′ in conventional fashion. The balloons are inflated to perform the angioplasty and to restore or substantially restore or at least partially restore the branches 310, 320 and 330 to their original diameters. The angioplasty balloons are then deflated and withdrawn.

A stent, such as a conventional stent 10 (or an ostial stent as described in the present application), is subsequently inserted and deployed as illustrated in FIG. 15. The stent 10 extends through the bifurcation from the incoming branch 310 to the outgoing branch 320. When so deployed, the second or outgoing branch 330 may be at least partially closed by the stent 10.

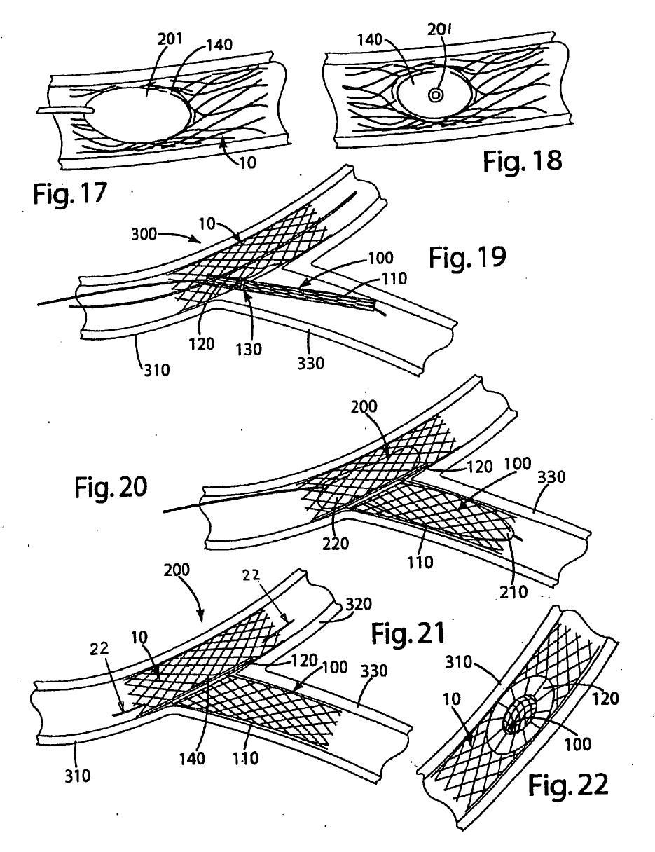

The guide wire 230′ is then withdrawn from the branch 330 and is advanced into the stent 10, through the wall (between the struts) of the stent 10, and into the other branch 330. A balloon, such as a conventional balloon 201 (or an ostial balloon as described in the present application), is then positioned on the wire 230′ and through the wall of the stent 10. If a new ostial stent is used, rather than a conventional stent, the balloon can be more easily inserted between the struts. The balloon 201 is then inflated or expanded. The procedure at this point is illustrated in FIGS. 16 and 17. The inflated balloon creates an opening 140 through the wall of the stent 10 by moving or separating/expanding the struts of the stent 10 as the balloon expands or inflates. The diameter of the opening 140 may be formed or established (via selective expansion/inflation of the balloon) to be approximately the same as the diameter of the branch 330.

The balloon 201 is then deflated as illustrated in FIG. 18. The struts do not readily retract to their original orientation or state when the balloon is deflated so that the opening 140 remains substantially the same size as created by the balloon. The balloon then is withdrawn from the stent 10 along the guide wire 230′.

The next step is to position an ostial stent 100 and balloon 200 (along guide wire 230′) in the opening 140 through the wall of the first stent 10. The result of this step is illustrated in FIG. 19. The distal or support portion 110 of the stent is thus located within the branch 330 while the proximal or flange portion 120 of the stent is located within the stent 10 and within the primary vessel 310.

The balloon 200 on which the stent 100 is mounted is then inflated as illustrated in FIG. 20. When the balloon is fully inflated, the distal portion 110 of the stent is expanded or deployed (via inflation of the distal portion 210 of the balloon) to support the branch 330, and the proximal portion 220 of the balloon forces the proximal portion 120 of the stent into a flange-like configuration against the wall of the first stent 10. The proximal portion and the distal portion of the balloon may be individually inflated or sequentially inflated or substantially simultaneously inflated or correspondingly inflated to expand the respective portions of the stent, such as in the manners described above. After the stent has been fully deployed at the vessel, the balloon 200 and both guide wires 230 and 230′ are withdrawn so that the final result is as illustrated in FIGS. 21 and 22.

Thus, a stent deployment system of the present invention may consist of two balloon delivery catheters, one which delivers and expands a stent in a primary vessel and a second which delivers and expands a stent in a vessel side branch. The first balloon catheter may have a balloon that allows for passage of a second guide wire to the side branch, and may allow the guide wire to be placed in the lumen of the side branch while dilation and stent deployment occurs in the main vessel (via inflation of the balloon). After the stent is placed and deployed in the main vessel, the balloon is withdrawn while the first and second guide wires preferably remain in place.

The second balloon delivery catheter is then threaded over the guide wire placed in the side branch, and the guide wire guides the second balloon and stent to the location where the second stent can be deployed at the ostium or opening of the side branch. The second balloon delivery catheter may consist of another balloon or two balloons (such as described above) that is/are inflated to deploy the side branch stent at the ostium of the side branch and through an opening in the first, already deployed, stent. After the second stent is deployed, the second balloon may be removed from the stent and side branch, preferably while the guide wires remain in place in the primary vessel and side branch vessel.

The resulting two-stent combination thus substantially or fully supports all areas in and through the entire bifurcation. The stents support the branches even at the junction of the branches without adversely expanding or affecting the junction of the branches or vessels. The method therefore provides previously unavailable treatment in a relatively simple but effective procedure.

V. One-Piece Bifurcated Stent

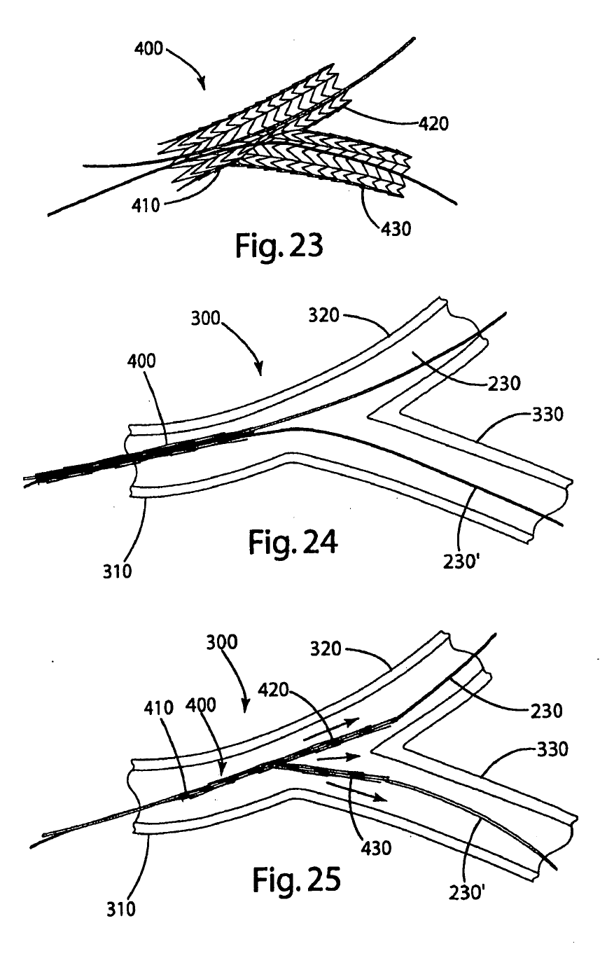

A one-piece, unitary stent for deployment in a bifurcation is illustrated in FIGS. 23-27 and generally designated 400. The stent 400 includes an inlet portion 410 and two outlet (or ostial) portions 420 and 430. The three portions form a generally Y-shaped stent. All of the portions 410, 420 and 430 are part of a single integrated whole. Methods and techniques fabricating the stent 400 will be apparent to those skilled in the art.

The bifurcated stent 400 is deployed in a bifurcation 300 such as illustrated in FIGS. 24-27. FIG. 24 shows the stent 400 mounted on the guide wires 230 and 230′. The guide wire 230 extends through portions 410 and 420, and the guide wire 230′ extends through portions 410 and 430.

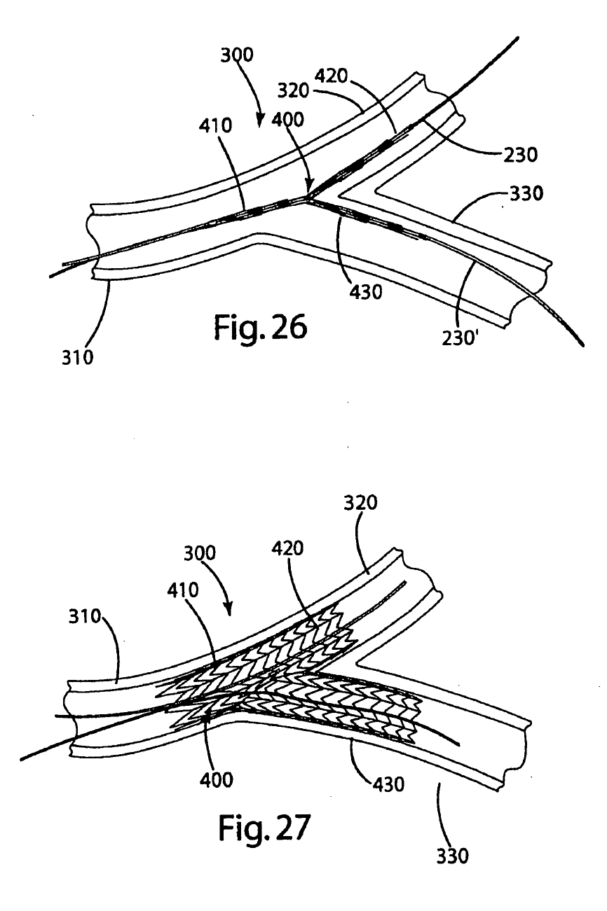

As shown in FIG. 25, the two portions 420 and 430 follow the guide wires 230 and 230′, respectively, into the respective branches 320 and 330. FIG. 26 shows the position of the stent 400 just prior to deployment, with the three stent portions 410, 420, and 430 positioned within the three branches 310, 320, and 330, respectively. FIG. 27 shows the stent 400 following deployment, with the three deployed or expanded stent portions 410, 420, and 430 supporting the respective bifurcation portions 310, 320 and 330.

The balloon (not shown) for deploying the stent 400 is preferably a Y-shaped balloon with portions of the balloon being located within respective portions of the stent. The balloon portions are inflatable to expand or deploy the portions of the stent at and within the Y-shaped vessel. The balloon portions may be part of a single balloon whereby the portions are inflatable together, or the balloon portions may be separate or individual chambers or portions that are individually inflatable or at least partially separately inflatable (such as due to separate chambers or different degrees of elasticity or expandability between the portions) to expand and deploy the stent. The construction and fabrication of such a balloon will be apparent to those skilled in the art upon review of the present application.

### VI. CONCLUSION

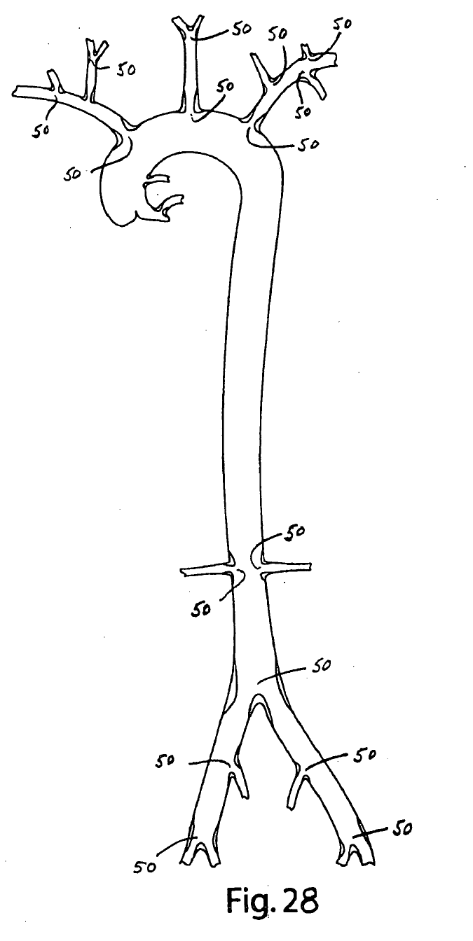

FIG. 28 is a schematic illustration of the aorta and primary arteries showing some of the possible locations 50 in which the stents of the present invention might be deployed. As can be seen, the possible locations are widespread and varying.

The above described stents and procedures enhance and expand cardiovascular procedures. The stents and procedures are highly effective and enable a variety of new areas, such as bifurcations, to be stented. The stents are less subject to movement and other subsequent complications.

Therefore, the present invention provides a stent and balloon configuration for expanding and supporting vessels, whereby the branch vessel is substantially supported by the stent and the stent is retained at the appropriate or desired location relative to the vessels. The stent may be deployed via an expandable or inflatable balloon and may be expandable to different degrees of radial expansion along its length. The balloon may inflate to different diameters or shapes along its length so as to selectively expand the stent to the different degrees of radial expansion. The balloon and stent combination of the present invention thus is configurable for various applications at an ostial branch at or near a primary vessel.

The above descriptions are those of current embodiments of the invention. Various alterations and changes can be made without departing from the spirit and broader aspects of the present invention, which is intended to be limited only by the scope of the appended claims, which are to be interpreted in accordance with the principles of patent law including the doctrine of equivalents.

The embodiments of the invention in which an exclusive property or privilege is claimed are defined as follows:

1. A cardiovascular stent and balloon combination comprising:
a stent, said stent comprising a first stent portion having at least a first degree of expandability and a second stent portion having at least a second degree of expandability greater than said first degree of expandability, said second stent portion terminating at one end of said stent; and
a balloon, said balloon comprising first and second balloon portions, said second balloon portion being inflatable to a larger diameter than a diameter of said first balloon portion, said balloon being positionable at least partially within said stent, whereby said first balloon portion is positioned at least partially within said first stent portion and said second balloon portion is positioned at least partially within said second stent portion.

2. The cardiovascular stent and balloon combination of claim 1 wherein said stent comprises a plurality of interconnected struts, a length of said struts of said first stent portion differing from a length of said struts of said second stent portion.

3. The cardiovascular stent and balloon combination of claim 2, wherein said struts of said second stent portion are longer than said struts of said first stent portion.

4. The cardiovascular stent and balloon combination of claim 2, wherein said stent comprises a third stent portion located between said first and second stent portions, said struts of said third stent portion being generally longitudinally oriented along said third stent portion, said third stent portion providing a transitional portion between said first and second stent portions.

5. The cardiovascular stent and balloon combination of claim 1, wherein said first balloon portion is expandable to a substantially ovoid shape and said second balloon portion is expandable to a substantially bulbous shape.

6. The cardiovascular stent and balloon combination of claim 1, wherein said first and second balloon portions comprise individual balloon portions that are individually inflatable via separate inflation tubes.

7. The cardiovascular stent assembly of claim 1, wherein said second stent portion is radially expandable so as to form a flange portion at an end of said first stent portion, said flange portion being oriented generally transverse to a longitudinal direction of said first stent portion.

8. The cardiovascular stent and balloon combination of claim 7, wherein said second balloon portion is inflatable to a generally bulbous form to establish said flange of said stent at said end of said first stent portion.

9. The cardiovascular stent and balloon combination of claim 8, wherein said first balloon portion is inflatable to a generally ovoid form to expand said first stent portion to a generally cylindrical form.

10. A method of treating a secondary cardiovascular vessel extending from a primary cardiovascular vessel, said method comprising the steps of:
providing a stent having first and second stent portions, said second stent portion being more expandable than said first stent portion;
providing a balloon having first and second balloon portions, said second balloon portion being expandable to a larger diameter than a diameter of said first balloon portion;
positioning said balloon at least partially within said stent so that said first balloon portion is at least partially within said first stent portion and said second balloon portion is at least partially within said second stent portion;
positioning said stent and balloon so that said first stent portion is located at a first vessel and said second stent portion is located at a second vessel;
inflating said second balloon portion to expand said second stent portion to form a stent flange;
engaging said stent flange with a wall of the second vessel with said first stent portion and said first balloon portion being within the first vessel; and
inflating said first balloon portion to expand said first stent portion to support the first vessel.

11. The method of claim 10, wherein providing a stent comprises providing a stent with struts of varying length between said first and second stent portions.

12. The method of claim 11, wherein said struts of said second stent portion are progressively longer struts along said second stent portion as said struts approach an end of said second stent portion that is opposite to said first stent portion.

13. The method of claim 11, wherein said stent comprises a transitional stent portion between said first and second stent portions, said transitional stent portion having struts that are arranged differently than said struts of said first and second stent portions.

14. The method of claim 10, wherein inflating said first balloon portion and inflating said second balloon portion comprises individually inflating said first balloon portion and said second balloon portion.

15. The method of claim 14, wherein after inflating said second balloon portion, said method includes moving said stent and said balloon until said flange of said stent engages the wall of the second vessel, said method including inflating said first balloon portion to expand said first stent portion to support the first vessel after said flange engages the wall of the second vessel.

16. The method of claim 15, wherein moving said stent and said balloon comprises advancing said stent and balloon until said flange of said stent engages the wall of the second vessel, said first stent portion comprising a distal portion of said stent and said second stent portion comprising a proximal portion of said stent relative to an entry point in the patient's body.

17. The method of claim 15, wherein moving said stent and said balloon comprises retracting said stent and balloon until said flange of said stent engages the wall of the second vessel, said first stent portion comprising a proximal portion of said stent and said second stent portion comprising a distal portion of said stent relative to an entry point in the patient's body.

18. A stent balloon comprising:
a first inflatable portion having a first diameter when inflated; and
a second inflatable portion having a second diameter when inflated, said second diameter being greater than said first diameter, whereby said stent balloon is capable of radially expanding a stent in which it is positioned to two different extents corresponding to said first and second diameters, said second inflatable portion being at least partially inflatable independently from said first inflatable portion.

19. The stent balloon of claim 18, wherein said first portion is ovoid shaped when inflated and said second portion is bulbously shaped when inflated.

20. The stent balloon of claim 18, wherein said first inflatable portion comprises a first chamber and said second inflatable portion comprises a second chamber, said first and second chambers being independently inflatable.

21. The stent balloon of claim 18, wherein said first and second inflatable portions are selectively inflatable so that one of said first and second inflatable portions is at least partially inflatable before the other of said first and second inflatable portions.

22. A cardiovascular stent comprising:
a first portion having at least a first degree of expandability;
a second portion having a second degree of expandability greater than said first degree of expandability, said second portion terminating at an end of said stent, said second portion being expandable to a progressively increased diameter along said second portion and toward said end of said stent, said second portion being expandable to form a flange that is generally transverse to a longitudinal direction of said first portion; and
a third portion between said first and second portions, said third portion having a third degree of expandability that is different from said first and second degrees of expandability.

23. The cardiovascular stent of claim 22, wherein said stent comprises first, second and third struts forming said first, second and third portions, respectively, a length of said first struts differing from a length of said second struts.

24. The cardiovascular stent of claim 23, wherein said second struts are longer than said first struts.

25. The cardiovascular stent of claim 23, wherein said second struts progressively increase in length along said second portion and toward said end of said stent.

26. The cardiovascular stent of claim 23, wherein said first and second struts are generally V-shaped or U-shaped struts that generally straighten as said first and second portions are radially expanded.

27. The cardiovascular stent of claim 26, wherein said third struts are arranged generally longitudinally along said third portion of said stent.
